(19) United States
(12) Plant Patent Application Publication
(10) Pub. No.: US 2011/0191923 P1
(43) Pub. Date: Aug. 4, 2011

(54) SPATHIPHYLLUM PLANT NAMED 'SWEET ROCCO'

(50) ;

(76) Inventor: Petrus Olsthoorn, Honselersdijk (NL)

(21) Appl. No.: 12/658,360

(22) Filed: Feb. 4, 2010

(51) Int. Cl. A01H 5/00

(52) U.S. Cl. PLT/364

(57) ABSTRACT

A new and distinct Spathiphyllum cultivar named ‘Sweet Rocco’ is disclosed, characterized by its early clump forming plant growth, and low growing dark green foliage. The new cultivar is a Spathiphyllum, typically suited for ornamental container use.

### LATIN NAME OF THE GENUS AND SPECIES

Spathiphyllum hybrid

### VARIETY DENOMINATION

‘Sweet Rocco’

### BACKGROUND OF THE INVENTION

The new cultivar is the result of a chance discovery in an existing population of Spathiphyllum hybrid plants. The new variety originated as a naturally occurring, whole plant mutation of the parent variety, the commercial variety, Spathiphyllum hybrid ‘Sweet Benito’ U.S. Plant Pat. No. 13,960. The new variety was discovered by the inventor, Petrus Olsthoorn in 2000 at a commercial greenhouse in Honselersdijk, the Netherlands.

After selecting and isolating the new cultivar, asexual reproduction of the new cultivar ‘Sweet Rocco’ was first performed in a commercial laboratory in Honselersdijk, the Netherlands by tissue culture in the winter of 2003. ‘Sweet Rocco’ has since produced several generations and has shown that the unique features of this cultivar are stable and reproduced true to type.

### SUMMARY OF THE INVENTION

The cultivar ‘Sweet Rocco’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘Sweet Rocco.’ These characteristics in combination distinguish ‘Sweet Rocco’ as a new and distinct Spathiphyllum cultivar:
1. Early development of basal clumps.
2. Foliage occurring heavily on the lower part of the plant.
3. Consistently dark foliage.

### PARENTAL COMPARISON

Plants of the new cultivar ‘Sweet Rocco’ are similar to the parent ‘Sweet Benito’ in most horticultural characteristics. However, ‘Sweet Rocco’ differs in producing foliage lower on the plant. Additionally, ‘Sweet Rocco’ produces darker foliage than ‘Sweet Benito.’

### COMMERCIAL COMPARISON

Plants of the new cultivar ‘Sweet Rocco’ are best compared to the parent variety.

### BRIEF DESCRIPTION OF THE PHOTOGRAPH

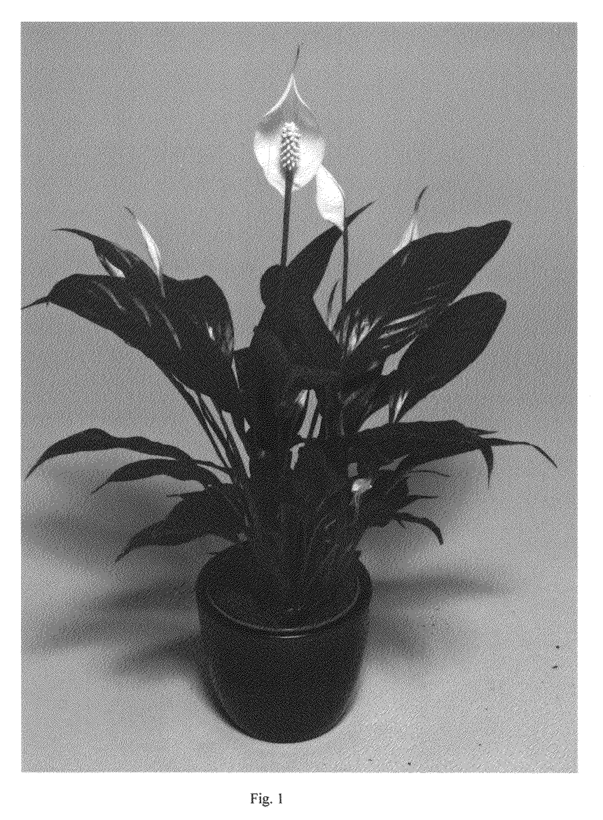

The accompanying photograph in FIG. 1 illustrates in full color a typical plant of ‘Sweet Rocco’ grown in a greenhouse in Honselersdijk, the Netherlands. This plant is approximately 6 months old, shown in a 12 cm pot. The photograph was taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart except where general terms of ordinary dictionary significance are used. The following observations and measurements describe ‘Sweet Rocco’ plants grown in a greenhouse in Honselersdijk, the Netherlands. Temperatures ranged from 18° C. to 20° C. at night to 19° C. to 20° C. during the day. No artificial light, photoperiodic treatments or chemical treatments were given to the plants. Measurements and numerical values represent averages of typical plant types.
Botanical classification: Spathiphyllum hybrid ‘Sweet Rocco’.
Age of the plant described: Approximately 6 months
Container size of the plant described: 12 cm commercial container.
Propagation:
Time to initiate roots: Approximately 7-14 days from an initial tissue culture plantlet at approximately 22° C.
Time to produce rooted plantlet: Approximately 4 weeks at approximately 22° C.
Root description: Moderately fibrous, not fleshy, moderately dense, colored near RHS Yellow-White 158D.
Plant:
Growth habit: Clump-forming, upright.
Plant shape: globular with flowers slightly above leaf plane.
Height: Approximately 30.8 cm to top of highest leaf, Approximately 47.0 cm to top of highest spathe.
Plant spread: Approximately 48.8 cm in a 12 cm pot.
Growth rate: low.
Branching characteristics: Leaves emerge direct from base of plant, forming clumps.
Number of clumps of leaves: Average 7.
Number of leaves per clump: Average 5.
Number of leaves per plant: Approximately 35.
Foliage:
Leaf:
   Arrangement.—alternate, growing directly from the soil.
   Average length (excluding petiole).—Approximately 19.9 cm.
   Average width.—Approximately 5.4 cm.
   Shape of blade.—narrow ovate to lanceolate.
   Aspect.—very slightly carinate, very slightly curved.
   Apex.—very long apiculate.
   Base.—attenuate.
   Margin.—entire, slightly undulate.
   Texture of top surface.—smooth, glossy.
   Texture of bottom surface.—smooth, moderately glossy.
   Color.—Young foliage upper side: Near in between R.H.S. Green N137A and 143A, but darker Young foliage under side: Near R.H.S. Yellow-Green 147B Mature foliage upper side: Near R.H.S. Green N137D Mature foliage under side: Near R.H.S. Green 137B.
Venation:
   Type.—pinnate.
   Venation coloration upper side.—Near R.H.S. Green 137B.
   Venation coloration under side.—Near R.H.S. Yellow-Green 145B.
Petiole (excluding geniculum):
   Length.—Approximately 11.6 cm.
   Width.—At geniculum: Approximately 0.3 cm Above clump: Approximately 0.6 cm.
   Color.—Near RHS Green 143A.
   Strength.—moderately strong.
Geniculum:
   Length.—Approximately 2.2 cm.
   Width.—Approximately 0.35 cm.
   Color.—Near RHS Green 138B.
Inflorescence:
   Arrangement.—inflorescences are carried slightly beyond and above the leaf plane.
   Flowering habit.—continuous.
   Quantity of spathes per plant.—On average 3 fully open spathes in good condition are open at one time.
   Natural flowering season.—late summer to midwinter.
   Time to flowering.—Approximately 4 months from a rooted young plant.
   Fragrance.—moderately strong, sweet and pleasant (typical Spathiphyllum scent).
   Self-cleaning or persistent.—persistent.
   Flower longevity.—Flowers stay in good condition approximately 3 weeks on the plant.
Spathe:
   Aspect.—upright.
   Length.—Approximately 9.9 cm.
   Width.—Approximately 4.2 cm.
   Shape.—ovate, concave.
   Margin.—entire.
   Apex.—apiculate to aristate.
   Base.—attenuate.
   Color.—Front when opening: Near RHS White 155C, main vein and tip near RHS Green 137B Back when opening: Near RHS White 155C, main vein and tip near RHS Green 137B Front when mature: Near RHS White N155A, base of main vein and tip near RHS Green 143B Back when mature: Near RHS White N155A, base of main vein and tip near RHS Green 143B Fading to: not fading.
Spadix:
   Shape.—oblong.
   Tip.—obtuse.
   Base.—obtuse.
   Length.—Approximately 3.0 cm.
   Width.—Approximately 1.2 cm.
   Color.—When opening: Near RHS Green-White 157C Mature: Near RHS Orange-White 159B.
   Quantity of flowers per spadix.—Approximately 75.
   Spadix flower arrangement.—spirally placed on spadix.
   Spadix flower diameter.—Approximately 0.35 cm.
   Spadix flower depth.—Approximately 0.25 cm.
Peduncle:
   Length.—Approximately 32.1 cm.
   Width.—Approximately 0.3 cm.
   Color.—Near RHS Yellow-Green 144A.
   Strength.—moderately strong.
Reproductive organs:
General description of visible parts: three stigmas are placed on top of each ovary (length of ovary approximately 0.25 cm), stigma diameter approximately 0.1 cm, colored Near RHS White N155D.
Other characteristics:
Seeds and fruits: No seeds/fruits observed.
Disease/pest resistance: Neither resistance nor susceptibility to the normal pests and diseases of Spathiphyllum has been observed.
Temperature tolerance: Indoor plant, no frost tolerance.

What is claimed is:

1. A new and distinct cultivar of Spathiphyllum hybrid plant named ‘Sweet Rocco’ as herein illustrated and described.
